(19) United States
(12) Patent Application Publication
(10) Pub. No.: US 2024/0307844 A1
(43) Pub. Date: Sep. 19, 2024

(54) METHOD FOR TREATING SURFACES OR GASEOUS MEDIA USING A FERROMAGNETIC GEL

(71) Applicant: COMMISSARIAT À L'ÉNERGIE ATOMIQUE ET AUX ÉNERGIES ALTERNATIVES, Paris (FR)

(72) Inventors: Alban Gossard, Avignon (FR); Fabien Frances, Saint-Julien De Peyrolas (FR); Hubert-Alexandre Turc, Les Angles (FR)

(21) Appl. No.: 18/279,636

(22) PCT Filed: Feb. 24, 2022
(86) PCT No.: PCT/FR2022/050337

§ 371 Date: Aug. 31, 2023

(30) Foreign Application Priority Data
Mar. 3, 2021 (FR) FR2102063

(51) Int. Cl. B01J 20/02; B01J 20/28; G21F 9/00; G21F 9/02

(52) U.S. Cl. CPC B01J 20/0296; B01J 20/28009; B01J 20/28047; G21F 9/002; G21F 9/02

(57) ABSTRACT

Disclosed a method for decontaminating a surface of a substrate or a method for decontaminating a gaseous medium using an inorganic ferromagnetic gel consisting of a colloidal solution comprising an inorganic thickening agent, a ferromagnetic compound and a solvent.

### TECHNICAL FIELD

One object of this invention is a method for decontaminating surfaces or gaseous media using a ferromagnetic decontamination gel.

Gels used in the method according to the invention are magnetisable gels, that is they are sensitive to a magnetic field created by a magnet.

Gels used in the method according to the invention are also generally suckable gels.

The present invention firstly relates to a method for decontaminating surfaces using a ferromagnetic decontamination gel.

The method for decontaminating surfaces according to the invention enables all kinds of surfaces, such as surfaces of metallic materials, plastic materials, minerals and vitreous materials, to be decontaminated.

The method for decontaminating surfaces according to the invention is especially applicable, among other things, for decontaminating surfaces of porous materials such as cementitious materials like mortar and concrete, bricks, plaster and natural or artificial stone.

The method for decontaminating surfaces according to the invention enables removal of all kinds of contaminant species (also called contaminants or pollutants) and in particular ionic, chemical, biological, nuclear or radioactive contaminant species.

The present invention also relates to a method for decontaminating gaseous media contaminated with contaminant species suspended in these gaseous media.

The gaseous medium may be air, in which case the method according to the invention is referred to as an atmospheric decontamination method.

Suspended contaminant species or contaminants—which may in particular be the above-mentioned contaminant species—may in particular be in the form of solid particles, liquid particles, or even in the form of molecular species.

More accurately, the method for decontaminating gaseous media according to the invention enables these suspended contaminants to be captured, trapped, pulled back and bound on a solid surface and, possibly, this surface to be decontaminated.

The technical field of the invention can, in general, be defined as that of the decontamination of surfaces of substrates or gaseous media, through a dry process, with a view to eliminating pollutants and contaminants found on these surfaces and/or under these surfaces (subsurface), that is embedded in the first layers of these substrates, or suspended in these gaseous media, and whose presence on and under these (subsurface) surfaces, that is embedded in the first layers of these substrates, or presence suspended in these gaseous media, is undesirable.

In other words, the technical field of the invention is the field of ionic, biological or chemical decontamination, disinfection, cleaning, degreasing of surfaces and depollution of solids and atmospheres.

The method for decontaminating surfaces or gaseous media according to the invention has numerous and diverse applications and concern many fields of activity, both industrial and domestic.

More specifically, the methods according to the invention can be used in the field of decontamination, dismantling of nuclear facilities and maintenance of nuclear facilities in the event of surface contamination.

### STATE OF PRIOR ART

“Suckable” decontamination gels are concentrated colloidal suspensions of mineral particles, for example silica or alumina particles. These mineral particles act as a viscous gelling agent for a liquid solution. The liquid solution of the gel contains one or more decontamination reagents and possibly co-viscosifiers, such as surfactants, making it possible in particular to adjust rheological properties. These various constituents are selected to formulate a gel that can be easily deposited or sprayed using adapted equipment.

Suckable gels make it possible to dispense with problems associated with the powdery nature of dry waste and enhance efficiency of the method implementing a gel.

These gels are currently being developed for two applications, that are: (1) the treatment, or more accurately the decontamination, of surfaces and (2) the treatment, or more accurately the decontamination, of gaseous media, in particular atmospheric decontamination.

### Surface Treatment Using Suckable Gels.

In the scope of surface treatment, gels are deposited or sprayed as thin layers onto the surface to be treated. By virtue of their rheological properties, the gels adhere to all types of partitions, even vertical ones, without flowing. The presence of a decontamination reagent in the formulation of gels enables a corrosive or dissolving action on the surface layers, releasing the embedded contamination. Contaminants are then solubilised and/or absorbed within the gels. Finally, the gels dry, fracture and produce non-powdery solid residues, called “flakes”, containing the contamination. These flakes are easily removed through a dry process, in particular by suction, hence the name ‘suckable’ gels. Some active ingredients, for example in so-called “BC” gels, also make it possible to break down biological and/or chemical contamination, rendering it harmless. Decontamination methods which implement these suckable gels are therefore dry surface decontamination methods, generating no liquid effluent and little dry solid residue. Indeed, these dry solid residues account on average for only a quarter of the mass of gel initially sprayed. In addition, these methods limit the time operators are exposed to radioactive contamination, because they are easy to implement by spraying and then sucking the dry residues, and because the operator's presence is not required while the gel is drying.

Surface treatment using suckable gels has been implemented within the scope of different applications.

Indeed, by adapting the formulation of gels, they can be used for operations of nuclear decontamination, surface decontamination or degreasing [1-2], biological surface decontamination [3] or removal of organic layers deposited onto the surface of a solid substrate [4].

Whatever the field of application, suckable gels are applied using a two-step method:

### First Step: Application of the Gel to the Surface to be Treated.

This application can be done in different ways:

Manually by trowelling. This technique consists in depositing the gel onto the surface with a spatula or trowel, and then spreading it like a coating. This technique is easy to perform on small, planar surfaces. However, it becomes difficult to apply when the surface is large or has a more complex geometry. For example, it is impossible or very difficult for the operator to spread the layer of gel inside a piping or duct. In addition, the presence of the operator in proximity to the contaminated surface is necessary, which can prove problematic in the scope of nuclear or biological decontamination operations in particular.

By spraying. This technique consists in depositing the gel using a spraying device, which facilitates and speeds up application of the gel layer, particularly on large planar surfaces. However, it is impossible for the operator to apply the gel to surfaces that are difficult to reach with the jet emitted by the spraying device, such as inside a duct or piping.

More generally, both trowel application and spray application of gels are satisfactory techniques when the surface is easily accessible, for example on a wall or floor. However, for hard-to-reach zones, such as inside a duct, it becomes quite complex, if not impossible, to apply gels, and liquid solutions or foams will thereby be preferred. But these solutions and foams generate contaminated liquid effluents, which should then be brought into compliance for treatment in Liquid Effluent Treatment Stations (LETS) or released into the environment.

### Second Step: Recovery of Residues, Dry Waste.

After application of the gels, action of the gels on the surface (decontamination, degreasing, etc.) and drying, the suckable gels form residues, being dry waste. These residues can then be recovered as follows:

Either manually, using a brush and shovel, and then transferring them to a suitable container. This manual operation is particularly arduous and repetitive for the operators and increases their exposure time to contamination, which is particularly problematic in the case of nuclear or biological decontamination operations especially.

Or using an electrical suction device, which has the advantage of avoiding close contact between the operator and the residue containing the contamination, and of putting the residue directly into an adapted container. However, sucking operations can resuspend particles. THE filters should therefore be used at the outlet of the suction device and will become additional secondary waste. In addition, recovering solid residues can prove to be complex if they are present in zones that are inaccessible to the suction device, which is often quite bulky.

### (2) Treatment of Gaseous Media and Atmospheres Using Suckable Gels.

Document [5] describes a method for decontaminating a gaseous medium such as air contaminated with suspended contaminant species, in which fine droplets are sprayed into this gaseous medium, thus forming a mist of an inorganic gel. The contaminant species are then taken up, captured by the gel droplets and the droplets will deposit and adhere to the partitions of the volume surrounding the gaseous medium (wall, floor, ceiling). The deposited droplets will thus form a gel layer that will eventually dry to form a dry residue containing the suspended contaminant species that have been taken up.

This method thus enables contaminants suspended in a gaseous medium to be captured and bound, but this method has several drawbacks:

Some droplets, sometimes very fine, take a long time to fall back down and can remain suspended for several hours, resulting in very lengthy decontamination operations.

Decontamination of narrow volumes, such as venting conduits, can be problematic because the place where the gel mist is formed is subject to restrictions inherent to the spraying device, which cannot access such volumes.

The deposition of gel droplets should sometimes be avoided on some sensitive surfaces, and the precision of spraying does not allow this.

It is sometimes complex to recover the final solid waste containing the contaminants, particularly if the droplets have been deposited in zones that are difficult to access.

The generation of very fine droplets can also lead to the formation of small solid waste which can re-suspend, particularly when suction recovery methods are used.

In the light of the foregoing, there is therefore a need for a method for decontaminating a surface, and for a method for decontaminating a volume of a gaseous medium, implementing a gel, in particular a suckable gel (whether this gel is applied by trowelling or spraying), which do not have the defects, drawbacks and disadvantages of methods of prior art as are set out above.

There is still a need for a decontamination method implementing a gel, which provides a solution to the problems of methods of prior art as have been mentioned above.

### DISCLOSURE OF THE INVENTION

This and other purposes are achieved, in accordance with the invention, by a method for decontaminating at least one surface of a substrate of a solid material, said surface being contaminated with at least one contaminant species located on said surface and/or below said surface (subsurface) in the first layers of the substrate, wherein at least one cycle comprising the following successive steps is carried out:
an inorganic ferromagnetic gel consisting of a colloidal solution comprising an inorganic viscosifier, a ferromagnetic compound and a solvent is applied to said surface, and then the gel applied to the surface is moved and spread at a distance using a magnet;
b) the gel is maintained on the surface at least for a time sufficient for the gel to destroy and/or inactivate and/or degrade and/or absorb the contaminant species, and for the gel to dry and form a dry, solid residue containing said contaminant species on the surface;
c) the dry, solid residue containing said contaminant species is moved and gathered on the surface using a magnet, and the dry, solid residue containing said contaminant species is recovered.

The substrate is therefore solid and the surface of this substrate is also solid.

Advantageously, the substrate of a solid material can be of a material selected from metals and metal alloys such as stainless steel, painted steels, aluminium and lead; polymers such as plastic materials or rubbers like polyvinyl chlorides (PVC), polypropylenes (PP), polyethylenes (PE), especially high density polyethylenes (HDPE), poly(methyl methacrylates) (PMMA), poly(vinylidene fluorides) (PVDF), polycarbonates (PC); glasses; cements and cementitious materials; mortars and concretes; plasters; bricks; natural or artificial stone; ceramics.

The contaminant species may be selected from ionic, chemical, biological, nuclear or radioactive contaminant species.

In particular, the contaminant species may be selected from all the contaminant species listed below in the description of the gel, as well as in the description of the method for decontaminating a gaseous medium.

In particular, the contaminant species can be an ionic contaminant species selected from monovalent and multivalent metal ions, in particular from toxic monovalent and multivalent metal ions such as chromium (VI), nickel (II), silver (I), cadmium (II), mercury (II), arsenic (III) and lead (II) ions.

The contaminant species may be a biological contaminant species selected from bacteria, fungi, yeasts, viruses, toxins, spores, in particular Bacillus anthracis spores, prions and protozoa.

In particular, the biological contaminant species may be selected from biotoxic species such as pathogenic spores, for example Bacillus anthracis spores, toxins, for example botulinum toxin or ricin, bacteria such as Yersinia pestis bacteria, prions, and viruses such as coronaviruses, vaccinia virus or haemorrhagic fever viruses, for example of the Ebola type.

The contaminant species may be selected from toxic chemical species, such as toxic gases, in particular neurotoxic or vesicant gases, in particular organophosphorus compounds, such as Sarin or GB agent, VX, Tabun or GA agent, Soman, Cyclosarin, diisopropyl fluoro phosphonate (DFP), Amiton or VG agent, Parathion, mustard gas or H agent or HD agent, Lewisite or L agent, T agent.

The contaminant species may be radioactive and/or chemically toxic, and/or toxic due to its shape and/or size.

The contaminant species which is toxic due to its shape and/or size may be a contaminant species in the form of solid particles such as microparticles or nanoparticles, for example in the form of fibres such as microfibres or nanofibres, in the form of nanotubes, or in the form of crystals such as nanocrystals.

In particular, the contaminant species may be asbestos.

Advantageously, the inorganic ferromagnetic gel can be applied to the surface to be decontaminated at a rate of from 100 g to 2000 g of gel per m² of area, preferably from 500 to 1500 g of gel per m² of area, even more preferably from 600 to 1000 g of gel per m² of area, which generally corresponds to a thickness of gel deposited onto the surface of between 0.5 mm and 2 mm.

Advantageously, during step b), a large thickness of gel, for example a thickness of from 2 mm to 2 cm, can be maintained on the surface using a magnet.

This makes it possible to increase depth of corrosion and effectiveness of decontamination.

Advantageously, during step a), the inorganic ferromagnetic gel is applied to said surface by spraying, brushing or with a trowel, and then the gel applied to the surface is moved and spread at a distance using a magnet.

Advantageously, the surface to be decontaminated may be a surface that is difficult, or even impossible, to reach by spraying, brushing or trowelling, for example the inner surface of a pipe or duct.

The inorganic ferromagnetic gel is then deposited onto the inner surface at the inlet of the pipe or duct and is then moved and spread on the inner surface using a magnet placed in the vicinity of the outer surface or on the outer surface of the pipe or duct.

Advantageously, drying is carried out at a temperature of from 1° C. to 50° C., preferably from 15° C. to 25° C., and under a relative humidity of from 20% to 80%, preferably from 20% to 70%.

Advantageously, the gel is maintained on the surface for a time of from 2 to 72 hours, preferably from 2 to 48 hours, more preferably from 4 to 24 hours.

Advantageously, the dry, solid residue is in the form of particles, for example flakes, with a size of 1 to 10 mm, preferably 2 to 5 mm.

Advantageously, the dry, solid residue containing said contaminant species is recovered using a magnet and/or by brushing and/or by suction, for example with a suction device provided with a magnet.

Advantageously, the cycle is repeated from 1 to 10 times, using the same gel for all the cycles, or using different gels for one or more cycles.

The invention also relates to a method for decontaminating a volume of a gaseous medium contaminated with suspended contaminant species, said volume of a gaseous medium being in contact with at least one surface of a solid substrate, said method comprising the following successive steps:
an inorganic ferromagnetic gel consisting of a colloidal solution comprising an inorganic viscosifier, a ferromagnetic compound and a solvent are sprayed into said volume of a gaseous medium of fine droplets, thus forming a mist;
b) the suspended contaminant species are captured, taken up by said droplets of ferromagnetic gel;
c) the droplets of the ferromagnetic gel containing the suspended contaminant species captured are moved under the action of a magnet until they are deposited and accumulate on a determined zone of said surface of the solid substrate;
d) the gel is maintained on the determined zone of the surface of the solid substrate at least for a time sufficient for the gel to dry and form a dry, solid residue containing the suspended contaminant species captured;
e) the dry, solid residue containing said suspended contaminant species captured is recovered.

For some aspects of the method according to the invention for decontaminating a gaseous volume, particularly with regard to steps a) and b), reference may be made to document [5] or to the corresponding international PCT application.

Advantageously, the gaseous medium may be air.

Advantageously, the volume of a gaseous medium may be an enclosed volume defined by partitions, such as a floor, a ceiling and walls, forming said surface, the mist of fine droplets fills the entire enclosed volume, and the fine droplets of ferromagnetic gel containing the suspended contaminant species captured are deposited onto at least one of the partitions, preferably on a lower partition such as a floor.

Advantageously, the fine droplets have a size, defined by their largest dimension, such as a diameter, of from 1 to 1000 μm, preferably from 5 to 200 μm.

The suspended contaminant species can be selected from all the contaminant species already listed above, upon describing the method for decontaminating a surface, and below upon describing the gel, with the general exception, however, of ionic species.

The suspended contaminant species may be in the form of solid particles, liquid particles or in the form of molecular species.

The contaminant species may be selected from chemical, biological, nuclear or radioactive contaminant species.

Suspended contaminant species may be radioactive contaminant species, and/or chemically toxic, and/or toxic due to their shape and/or size.

Suspended contaminant species which are toxic due to their shape and/or size may be selected from contaminant species in the form of solid particles such as microparticles or nanoparticles, for example in the form of fibres such as microfibres or nanofibres, in the form of nanotubes, or in the form of crystals such as nanocrystals.

In particular, the suspended contaminant species may be selected from metals and metalloids in metal, metalloid or ionic form, preferably from so-called “heavy metals”, and toxic metals and metalloids in metal, metalloid or ionic form; compounds of these metals and metalloids such as organometallic compounds, metal salts, metal oxides, metal carbides, etc.; ceramics; wood; cereals; flour; asbestos; and glasses, for example in the form of glass wool.

In particular, the suspended contaminant species may be asbestos.

Advantageously, the dry, solid residue containing said suspended contaminant species captured can be recovered using a magnet and/or by brushing and/or by suction, for example with a suction device provided with a magnet.

Advantageously, the magnet is an electromagnet.

Indeed, the use of an electromagnet during step a) or step c) of the method for decontaminating at least one surface or during step e) of the method for decontaminating a volume of a gaseous medium, that is upon spreading the gel or upon recovering the solid dry residues, facilitates the decontamination method because:

The electromagnet can be switched off, inactivated until it reaches the defined zone of interest, and then activated only to move the gel. This avoids parasitic magnetisations which could be harmful, depending on where the gel is implemented.

Similarly, the electromagnet can be activated to recover waste and solid residues and then deactivated to release the solid waste and transfer it to a suitable container.

Advantageously, the magnet is moved by remote handling or teleoperation.

Indeed, spreading and recovery of the gel using a magnet can be carried out by an operator but can also be done by remote handling. Indeed, in the case of areas that are difficult to access because they are heavily contaminated, it is much easier to deposit the gel, spread it out and then recover the solid waste using a magnet by remote handling than implementing suckable gels by spraying and then sucking.

The method according to the invention, both for decontaminating surfaces and for decontaminating gaseous media, implements a specific gel, which is an inorganic ferromagnetic gel consisting of a colloidal solution comprising an inorganic viscosifier, a ferromagnetic compound and a solvent.

By “ferromagnetic compound”, it is meant a compound sensitive to the action of a magnetic field, more accurately a compound sensitive to the action of a magnetic field generated by a magnet, in particular a compound which can be attracted by a magnet or forming a permanent magnet.

The gel implemented in the method according to the invention, because it contains a ferromagnetic compound, may be called a “ferromagnetic gel”.

The gel implemented in the method according to the invention can also be called a “magnetisable gel” because, since it contains a ferromagnetic compound, it is sensitive to the action of a magnetic field generated by a magnet and can be attracted and moved by a magnet.

The term “gel” is perfectly clear to the person skilled in the art and has a widely accepted meaning.

However, in general, a gel can be considered to have a viscosity greater than or equal to 0.1 Pa·s.

Generally, the amount of solvent is at least 20% by mass, in relation to the mass of the gel, as otherwise a gel is generally not obtained but rather a very pasty and unbound mixture which cannot be moved correctly by a magnet is obtained.

The gel implemented in the method according to the invention is termed an inorganic, mineral gel in that it comprises an inorganic, mineral viscosifier.

Generally, the gel contains only this inorganic, mineral viscosifier as a viscosifier, and does not contain any organic viscosifier.

The gel implemented in the method according to the invention can generally be defined as a “suckable gel”.

The term “suckable gel” is commonly used in this field of technology, and has a widely accepted meaning, as described below.

A suckable gel is intrinsically different from a gel that is not suckable.

The presence of a ferromagnetic compound in the decontamination gel implemented in the method according to the invention is one of the fundamental characteristics which differentiates the method according to the invention from methods of prior art.

A decontamination method implementing a decontamination gel in which a ferromagnetic compound is incorporated is neither described nor suggested in documents of prior art, as represented in particular by documents [1] to [5] cited above.

The gel implemented in the method according to the invention can therefore be defined as a conventional suckable gel to which a ferromagnetic compound is added.

The method according to the invention, which implements a particular decontamination gel containing a ferromagnetic compound, makes it possible, in particular because of the presence of this ferromagnetic compound in the gel, to overcome all the drawbacks of methods for decontaminating surfaces and gaseous media using suckable gels of prior art, and to provide a solution to the problems posed by these methods.

Unlike methods of prior art, the presence of a ferromagnetic compound in the gel implemented in the method according to the invention means that the gel can be moved easily under the action of a magnetic field, for example by attracting the ferromagnetic gel with a magnet. The fact that the ferromagnetic gel can be easily displaced by the action of a magnetic field, for example a magnetic field created by a magnet, brings numerous advantages—set out herein—within the scope of methods for decontaminating surfaces and gaseous media and makes it possible to overcome problems that these methods had.

In addition, the solid residues generated by drying the gel can also be easily displaced and recovered under the action of a magnetic field, for example a magnetic field created by a magnet, which also brings many advantages—set out herein—within the context of methods for decontaminating surfaces and gaseous media, and makes it possible to overcome the problems that these methods had.

The suckable gels used in the method according to the invention have all the advantageous properties known to suckable gels, and these advantageous properties are in no way affected by the addition to these gels of a ferromagnetic compound, which itself provides other advantageous properties.

In particular, the addition of this ferromagnetic compound to the gel does not in any way alter the steps of preparing the suckable gel.

Ferromagnetic compounds are easy to use and are incorporated into the gel formulation implemented in the method according to the invention in the form of particles during the stirring step upon preparing gel according to the invention.

It is also important to notice that the addition of ferromagnetic compounds does not significantly modify the physico-chemical behaviour of the gel, particularly with regard to rheology and fracturing during drying.

As a result, the gel formulation can be easily adapted to the desired gel texture depending on the intended application. The gel formulation is adapted to the acidic, basic and solvent media of decontamination gels.

The method according to the invention has a number of advantages related to the presence of ferromagnetic compounds in the decontamination gels implemented by the method according to the invention. These advantages are among others:

### For Application of the Gel to a Contaminated Surface:

The possibility of moving the gel using a permanent magnet or an electromagnet. This operation can be carried out:
either remotely, by moving the magnet above the gel to form a homogeneous layer,
or through a partition by placing the magnet on the opposite face of the surface to be decontaminated, which is extremely advantageous because it can spread the gel inside a duct, for example.

Generally speaking, the possibility of moving the gel using a magnet makes it possible to deposit a layer of gel on surfaces that are difficult to access or even impossible to reach, particularly by spraying.

The use of a magnet can also make it possible to maintain the gel on a specific zone (for example a hot spot on a surface) with a significant thickness and avoid any flowing. This can enable the surface to be decontaminated more deeply.

### For Decontamination of Atmospheres:

The possibility of attracting fine droplets of gel to capture suspended aerosols with a magnet. This can speed up an atmosphere decontamination operation by causing the finest droplets to fall, which might otherwise remain suspended for several hours.

The possibility of moving a cloud of gel droplets by attracting them with a magnet. This can be advantageous for decontaminating the inside of a narrow volume, such as a venting conduit.

For Recovering Solid Waste after the Gel has Dried:

Recovering gel residues with a suction device provided with a magnet allows the solid waste to be recovered without the need for large streams (reduced extraction requirements) and THE filters.

Thereby, this also avoids any possible resuspension of powder particles due to the streams involved.

Residues can also be recovered simply, and without suction, by using an electromagnet to gather residues on the magnetised face, and then by deactivating the electromagnet above a “dustbin” container, the residues are detached and fall directly into the container.

The speed with which the residues can be recovered by simply passing close to the surface means that the operators' exposure time is reduced.

The use of a magnet to recover solid waste is more easily achieved by remote handling or teleoperation than the use of a suction device.

Advantageously, the colloidal solution further comprises one or more components selected from the following components:
a surfactant;
an active decontamination agent;
a getter to trap gaseous contaminant species, in particular toxic or explosive gaseous contaminant species, such as hydrogen;
a superabsorbent polymer;
a contaminant species extractant.

Preferably, the colloidal solution comprises, preferably consists of:
1% to 40% by mass, preferably 5% to 30% by mass, even more preferably 5% to 25% by mass, better still 8% to 20% by mass, relative to the mass of the gel, of at least one inorganic viscosifier;
0.1% to 40% by mass, preferably 5% to 30% by mass, relative to the mass of the gel, of at least one ferromagnetic compound;
optionally, 0.05% to 2% by mass, relative to the mass of the gel, of at least one surfactant;
optionally, 0.05 to 10 mol/L of gel, preferably 0.1 to 5 mol/L of gel, even more preferably 1 to 2 mol/L of gel, of at least one active decontamination agent;
optionally, 0.1% to 5% by mass, relative to the mass of the gel, of at least one getter to take up, trap gaseous contaminant species, in particular toxic or explosive gaseous contaminant species, such as hydrogen;
optionally, 0.05% to 5% by mass, preferably 0.05% to 2% by mass, relative to the mass of the gel, of at least one superabsorbent polymer;
optionally, 0.1% to 5% by mass, relative to the mass of the gel, of at least one contaminant species extractant;
and the remainder solvent, the amount of solvent being at least 20% by mass, relative to the mass of the gel.

The sum of the percentages by mass of all the components making up the gel is obviously 100% by mass.

By “solvent residue”, it is meant that the solvent is always present in the colloidal solution and that the amount of solvent is an amount such that, when it is added to the amounts of the components of the colloidal solution other than the solvent (whether these components are obligatory or optional components mentioned above, or other optional additional components mentioned or not mentioned), the total amount of all the components of the colloidal solution is 100% by mass.

It should be noted that it is not necessary for the gel to contain one or more of the optional components mentioned above. Indeed, a gel comprising only the solvent, the viscosifier and the ferromagnetic compound can be used successfully, and makes it possible to achieve the effects and advantages listed above.

In other words, preferably the colloidal solution comprises, preferably consists of:
1% to 40% by mass, preferably 5% to 30% by mass, even more preferably 5% to 25% by mass, better still 8% to 20% by mass, relative to the mass of the gel, of at least one inorganic viscosifier;
0.1% to 40% by mass, preferably 5% to 30% by mass, relative to the mass of the gel, of at least one ferromagnetic compound;
and one or more component(s) selected from the following components, in the following proportions:
optionally, 0.05% to 2% by mass, relative to the mass of the gel, of at least one surfactant;
optionally, 0.05 to 10 mol/L of gel, preferably 0.1 to 5 mol/L of gel, even more preferably 1 to 2 mol/L of gel, of at least one active decontamination agent;
optionally, 0.1% to 5% by mass, relative to the mass of the gel, of at least one getter to take up, trap gaseous contaminant species, in particular toxic or explosive gaseous contaminant species, such as hydrogen;
optionally, 0.05% to 5% by mass, preferably 0.05% to 2% by mass, relative to the mass of the gel, of at least one superabsorbent polymer;
optionally, 0.1% to 5% by mass, relative to the mass of the gel, of at least one contaminant species extractant;—and the remainder solvent, the amount of solvent being at least 20% by mass, relative to the mass of the gel.

In other words, or preferably, the colloidal solution comprises, preferably consists of:
1% to 40% by mass, preferably 5% to 30% by mass, even more preferably 5% to 25% by mass, better still 8% to 20% by mass, relative to the mass of the gel, of at least one inorganic viscosifier;
0.1% to 40% by mass, preferably 5% to 30% by mass, relative to the mass of the gel, of at least one ferromagnetic compound;
and the remainder solvent, the amount of solvent being at least 20% by mass, relative to the mass of the gel.

The presence of a surfactant in the gel implemented in the method according to the invention favourably and noticeably influences rheological properties of the gel. In particular, this surfactant helps the gel to regain its viscosity following application or spraying, fogging or atomisation in the form of fine droplets, and avoids the risk of spreading out or flowing out when the gel is applied or deposited onto vertical surfaces and ceilings.

The presence of at least one active decontamination agent in the gel enables contaminant species to be removed, destroyed, inactivated, killed or extracted.

The presence of at least one contaminant species extractant in the gel facilitates capture and binding of contaminant species.

The presence of at least one superabsorbent polymer in the gel makes it possible to improve decontamination performance of the gels on porous surfaces, particularly cementitious materials, when the polluting contaminants have penetrated deep into the pores.

The gel implemented in the method according to the invention is a colloidal solution, which means that the gel according to the invention contains solid inorganic, mineral particles of viscosifier, the elementary, primary particles of which are generally between 2 nm and 20 μm in size, preferably between 2 nm and 200 nm.

Due to the implementation of a generally exclusively inorganic viscosifier, without any organic viscosifier, the organic matter content of the gel implemented according to the invention is generally less than 4% by mass, preferably less than 2% by mass, which is an advantage of the gels implemented according to the invention.

These solid, mineral or inorganic particles act as a viscosifier to enable the solution, for example the aqueous solution, to gel.

Advantageously, the inorganic viscosifier can be selected from metal oxides such as aluminas, metalloid oxides such as silicas, metal hydroxides, metalloid hydroxides, metal oxyhydroxides, metalloid oxyhydroxides, aluminosilicates, clays such as smectite, and mixtures thereof.

In particular, the inorganic viscosifier may be selected from aluminas (Al₂O₃) and silicas (SiO₂).

The inorganic viscosifier may comprise only one silica or alumina or a mixture thereof, namely a mixture of two or more different silicas (SiO₂/SiO₂ mixture), a mixture of two or more different aluminas (Al₂O₃/Al₂O₃ mixture), or even a mixture of one or more silicas with one or more aluminas (SiO₂/Al₂O₃ mixture).

Advantageously, the inorganic viscosifier can be selected from pyrogenated silicas, precipitated silicas, hydrophilic silicas, hydrophobic silicas, acidic silicas, basic silicas such as Tixosil® 73 silica, marketed by the company Rhodia, and mixtures thereof.

Among the acidic silicas, particular mention may be made of the pyrogenated or fumed silicas “Cab-O-Sil” ® M5, H5 or EH5, marketed by the company CABOT®, and pyrogenated silicas marketed by the company EVONIK INDUSTRIES®° as AEROSIL®°.

Among these fumed silicas, AEROSIL®° 380 silica, having a specific surface area of 380 m²/g, which offers maximum viscosity properties for a minimum mineral filler, is even more preferred.

The silica used can also be a so-called precipitated silica obtained, for example, by the wet process by mixing a solution of sodium silicate and an acid. Preferred precipitated silicas are marketed by the company EVONIK INDUSTRIES® as SIPERNAT® 22 LS and FK 310 or by the company RHODIA® as TIXOSIL® 331, the latter being a precipitated silica with an average specific surface area of between 170 and 200 m²/g.

Advantageously, the inorganic viscosifier consists of a mixture of a precipitated silica and a pyrogenated silica.

The alumina may be selected from calcined alumina, ground calcined alumina and mixtures thereof.

Advantageously, the inorganic viscosifier can consist of one or more alumina(s) generally accounting of from 5% to 30% by mass relative to the total weight of the gel.

In this case, the alumina(s) is (are) preferably at a concentration of from 8% to 17% by mass relative to the total mass of the gel to ensure that the gel dries at a temperature of between 20° C. and 50° C. and at a relative humidity of between 20% and 60% on average in 30 minutes to 5 hours.

Advantageously, the alumina(s) may be selected from pyrogenated aluminas, preferably from fine-grain pyrogenated aluminas.

By way of example, mention may be made of the product sold by the company EVONIK INDUSTRIES® under the trade name “Aeroxide Alumina C”, which is fine pyrogenated alumina.

The nature of the inorganic mineral viscosifier, particularly but not exclusively when it consists of one or more alumina(s), unexpected influences drying of the gel implemented in the method according to the invention and the particle size of the residue obtained.

This is also the case when the viscosifier consists of one or more silicas and more generally of one or more oxides of metals or metalloids described above.

This property is not exclusive to alumina but is a common, general property of suckable gels.

Indeed, the dry gel is in the form of particles of controlled size, more accurately millimetre-sized solid flakes, the size of which generally ranges from 1 to 10 mm, preferably from 2 to 5 mm, especially by virtue of the aforementioned compositions, in particular, but not only, when the viscosifier consists of one or more alumina(s).

Here again, this is also the case when the viscosifier consists of one or more silicas and, more generally, of one or more oxides of metals or metalloids described above.

It should be noted that the size of the particles generally corresponds to their largest dimension.

In other words, the solid mineral particles of the gel implemented according to the invention, for example of the silica or alumina type, in addition to their role as viscosifiers, also play a fundamental role upon drying the gel as they ensure fracturing of the gel to end up with a dry waste product in the form of flakes.

The gel implemented in the method according to the invention may contain an active decontamination agent.

This active decontamination agent may be any active decontamination agent enabling a contaminant species to be removed, whatever the nature of this contaminant species: whether this contaminant species is chemical, biological or even nuclear, radioactive—in other words, this decontamination agent may be any “NRBC” (Nuclear, Biological, Radiological, Chemical) decontamination agent, or whether this contaminant species is organic or mineral, liquid or solid.

The gel implemented in the method according to the invention can therefore contain an active biological or chemical or even nuclear or radioactive decontamination agent.

The active decontamination agent may also be a degreasing, stripping agent, or a corrosive agent, in order to eliminate any contaminant species present on the surface of a substrate, and/or below the surface (subsurface), that is embedded in the first layers of the substrate (from the surface), deep into the substrate.

Some active decontamination agents can perform several decontamination functions simultaneously.

By biological decontamination agent, which can also be termed a biocidal agent, it is meant any agent which, when brought into contact with a biological species and especially a toxic biological species, is likely to inactivate or destroy the same.

By biological species, it is meant any type of micro-organism such as bacteria, fungi, yeasts, viruses, toxins, spores, in particular Bacillus anthracis spores, prions and protozoa.

Biological species removed, destroyed or inactivated by the gel used in the method according to the invention are essentially biotoxic species such as pathogenic spores like Bacillus anthracis spores, toxins like botulinum toxin or ricin, bacteria like Yersinia pestis bacteria, and viruses like coronaviruses, vaccinia virus or haemorrhagic fever viruses for example of the Ebola type.

By chemical decontamination agent, it is meant any agent which, when brought into contact with a chemical species and in particular a toxic chemical species, is likely to destroy or inactivate the same.

The chemical species which are removed by the gel implemented in the method according to the invention are especially toxic chemical species such as toxic gases, in particular neurotoxic or vesicant gases.

These toxic gases are in particular organophosphorus compounds, including Sarin or agent GB, VX, Tabun or agent GA, Soman, Cyclosarin, diisopropyl fluoro phosphonate (DFP), Amiton or agent VG and Parathion. Other toxic gases are mustard gas or agent H or agent H D, Lewisite or agent L, agent T.

The active decontamination agent, for example the active biological or chemical decontamination agent, can be selected from bases such as sodium hydroxide, potassium hydroxide, and mixtures thereof; acids such as nitric acid, phosphoric acid, hydrochloric acid, sulphuric acid, hydrogen oxalates such as sodium hydrogen oxalate, and mixtures thereof; oxidising agents such as peroxides, permanganates, persulphates, ozone, hypochlorites such as sodium hypochlorite, cerium IV salts, and mixtures thereof; quaternary ammonium salts such as hexadecylpyridinium (cetylpyridinium) salts, such as hexadecylpyridinium (cetylpyridinium) chloride; reducing agents; and mixtures thereof.

For example, the active decontamination agent may be a disinfectant such as bleach, which provides the gel with decontamination, biological and/or chemical decontamination properties.

Some active decontamination agents may fall into several of the categories defined above.

Thus, nitric acid is an acid, but it is also an oxidising and corrosive agent.

The active decontamination agent, such as a biocidal agent, is generally used at a concentration of 0.05 to 10 mol/L of gel, preferably 0.1 to 5 mol/L of gel, even more preferably 1 to 2 mol/L of gel, in order to guarantee decontamination power, for example a capacity to inhibit biological, especially biotoxic, species, compatible with the drying time of the gel, and to ensure, for example, that the gel dries at a temperature of between 20° C. and 50° C. and at a relative humidity of between 20% and 60% on average in 30 minutes to 5 hours.

In order to achieve total effectiveness, including in the most unfavourable temperature and humidity conditions with regard to drying time, the gel formulation supports different concentrations of active agent. Indeed, it can be noticed that increasing the concentration of decontamination agent, particularly acidic or basic decontamination agent, increases the efficiency of the method.

The active decontamination agent can be an acid or a mixture of acids. These acids are generally selected from mineral acids such as hydrochloric acid, nitric acid, sulphuric acid and phosphoric acid.

Acids, such as nitric acid, have a corrosive action that enables a surface layer of a solid substrate containing contaminant species to be removed (see example 5).

In other words, acids are generally used for their corrosive power to release contamination embedded in the first layers (from the surface) of a contaminated substrate, particularly a metal substrate.

The acid(s) is (are) preferably present at a concentration of 0.5 to 10 mol/L, more preferably 1 to 10 mol/L, better still 3 to 6 mol/L to ensure that the gel dries generally at a temperature of between 20° C. and 50° C. and at a relative humidity of between 20% and 60% on average in 30 minutes to 5 hours.

For this type of acidic gel, the inorganic viscosifier is preferably silica or a mixture of silicas.

Alternatively, the active decontamination agent, for example the active biological decontamination agent, may be a base, preferably a mineral base, preferably selected from soda, potash and mixtures thereof.

In the case of such a basic gel formulation, the gel implemented in the method according to the invention has, in addition to the decontamination action, a degreasing action which also makes it possible to remove possible contaminant species on the surface of the substrate.

As has already been mentioned above, in order to achieve total effectiveness, including under the most unfavourable climatic conditions with regard to gel drying time, the gel implemented in the method according to the invention can have a wide range of concentration(s) of basic decontamination agent(s).

Indeed, increasing the concentration of the basic decontamination agent such as NaOH or KOH, which generally acts as a biocidal agent, makes it possible to considerably increase the inhibition rates of biological species, as has been demonstrated for Bacillus thuringiensis spores.

The base is advantageously present in a concentration of less than 10 mol/L, preferably between 0.5 and 7 mol/L, even more preferably between 1 and 5 mol/L, better still between 3 and 6 mol/L, to ensure that the gel dries at a temperature of between 20° C. and 50° C. and at a relative humidity of between 20% and 60% on average in 30 minutes to 5 hours.

For this type of alkaline, basic gel, the inorganic viscosifier is preferably an alumina or a mixture of aluminas.

The decontamination agent, particularly when it is a biological decontamination agent, is preferably sodium hydroxide or potassium hydroxide.

With regard, for example, to spore inhibition kinetics and gel drying times as a function of temperature, the active decontamination agent, especially in the case of a biocidal agent, will preferably be sodium hydroxide at a concentration of between 1 and 5 mol/L.

For biological decontamination, preferred decontamination agents are the combination of a mineral base and an oxidising agent as is described in document [3], to the description of which reference may be made.

The gel may also contain a surfactant or a mixture of surfactants.

Advantageously, the surfactant may be selected from surfactants having one or more of wetting properties, emulsifying properties and detergent properties; and mixtures thereof.

The surfactant may be selected from the group consisting of alcohol alkoxylates, alkyl aryl sulphonates, alkyl phenol ethoxylates, block copolymers based on ethylene oxide and/or propylene oxide, ethoxylated alcohols, ether phosphates, light and heavy ethoxylated acids, glycerol esters, imidazolines, quaternary ammonium salts (quats), alkanolamides, amine oxides, and mixtures thereof.

The surfactants are preferably block copolymers marketed by the company BASF® under the name PLURONIC®.

Pluronics® are block copolymers of ethylene oxide and propylene oxide.

Pluronics® are generally preferred for biological decontamination gels.

Other surfactants are preferred for decontamination gels requiring a corrosive action, such as acidic gels. In this respect, reference can be made to document [1].

Surfactants influence rheological properties of the gel, especially the thixotropic nature of the product and its recovery time, and prevent flowing from occurring.

The surfactants also make it possible to control adhesion of the dry waste and to control size of the dry residue flakes to ensure that the waste is not powdery.

Advantageously, the ferromagnetic compound is selected from ferromagnetic metals such as iron, cobalt and nickel; ferromagnetic alloys such as Heusler alloys, and alloys forming permanent magnets such as rare earth permanent magnets such as neodymium or dysprosium or cobalt permanent magnets; and ferrites.

The ferromagnetic compound is generally in the form of particles, for example spherical or spheroidal particles.

These particles generally have a size (defined by their largest dimension such as their diameter) of from 2 nm to 20 μm.

Advantageously, the getter is selected from solids with a very large specific surface area that contain active surface sites, such as porous carbons, for example activated carbons; particles of cage materials, such as zeolite and MOF particles; and mineral oxide particles capable of reacting with contaminant species to be trapped and more specifically with hydrogen, such as manganese oxides (MnO₃, Mn₂O₃, Mn₂O₄) and noble metal oxides, for example Ag₂O or RuO₂.

Advantageously, the contaminant species extractant is selected from inorganic adsorbents such as zeolites, clays, phosphates such as apatites, titanates such as sodium titanates, and ferrocyanides and ferricyanides.

This possible extractant such as a zeolite or a clay can be used in the case where the contaminant species is a radionuclide, but this possible extractant can also be used in the case of contaminant species other than radionuclides, as for example metals, such as toxic metals or heavy metals.

By “superabsorbent polymer”, also known as “SAP”, it is generally meant a polymer capable, in the dry state, of spontaneously absorbing at least 10 times, preferably at least 20 times, its weight of aqueous liquid, in particular water and in particular distilled water.

Such superabsorbent polymers are especially described in document WO-A1-2013/092633 [7], to which reference may be made.

The solvent for the gel implemented in the method according to the invention is generally selected from water, organic solvents such as terpenes and alcohols and mixtures thereof.

A preferred solvent is water, and in this case the solvent therefore consists of water, comprising 100% water.

Alternatively, the solvent for the gel implemented in the method according to the invention may comprise, preferably consists of one or more organic solvents such as terpenes, in particular d-limonene.

The use of an organic solvent in the gel implemented in the method according to the invention will make it possible to remove organic layers (for example paint, coating or bitumen stain) containing contaminant species from the surface of a solid substrate.

Other characteristics and advantages of the invention will become clearer from the following detailed description, made for illustrating and non-limiting purposes, in conjunction with the appended drawings.

### BRIEF DESCRIPTION OF THE DRAWINGS

FIG. 1 is a schematic view of an example of a surface of a solid substrate—namely the surface of the inside of a piping—which can be treated by the method for decontaminating a surface of a solid substrate according to the invention, using an inorganic ferromagnetic gel.

FIG. 2A and

FIG. 2B and

FIG. 2C and

FIG. 2D are schematic cross-section views showing successive steps of a method according to the invention for decontaminating a surface of a solid substrate using an inorganic ferromagnetic gel.

FIG. 3A and

FIG. 3B are schematic cross-section views showing successive steps in a method according to the invention for decontaminating a volume of a gaseous medium contaminated with suspended contaminant species using an inorganic ferromagnetic gel.

FIG. 3C and

FIG. 3D are schematic views illustrating recovery, using an electromagnet, of the dry and solid waste obtained after a decontamination operation carried out in accordance with the method according to the invention, using a ferromagnetic gel.

FIG. 4 is a graph showing the course of viscosity (in Pa·s) as a function of the shear rate (in l/s), for gels referred to as “Gels 4, 9, 10 and 12” prepared in example 1 (see example 2).

FIG. 5 is a graph showing the course of shear stress (in Pa) as a function of strain (in %) for gels referred to as “Gels 4, 9, 10 and 12” prepared in example 1 (see example 2).

FIG. 6A and

FIG. 6B and

FIG. 6C and

FIG. 6D are photographs showing, respectively, spreading with a magnet of the gels referred to as “Gels 1, 2, 3, and 4” prepared in example 1 (see example 3).

FIG. 7A and

FIG. 7B and

FIG. 7C are photographs showing the magnetisable gel referred to as “Gel 13” prepared in example 1, deposited before drying (FIG. 7A), after drying (FIG. 7B), and the recovery of the dry gel residues after drying using a magnet (FIG. 7C) (see example 4).

FIG. 8A and

FIG. 8B and

FIG. 8C and

FIG. 8D are photographs showing corrosion of a steel plate surface by the magnetisable gel referred to as “Gel 13” prepared in example 1. FIG. 8A shows the initial plate without gel, FIG. 8B shows the plate with deposited gel, FIG. 8C shows the plate with deposited gel after drying, and FIG. 8D shows the plate after removal of the solid residues of dried gel (see Example 5).

### DETAILED DISCLOSURE OF PARTICULAR EMBODIMENTS

The gel implemented in the decontamination method according to the invention (both for a surface and for a volume of a gaseous medium) can be easily prepared at ambient temperature.

For example, the gel implemented in the method according to the invention can be prepared by adding, preferably gradually, the ferromagnetic compound to the solvent such as water, preferably deionised water, or to a mixture of the solvent and one or more components selected from among the components already listed above, namely: a surfactant; a superabsorbent polymer; an active decontamination agent; a getter; a contaminant species extractant.

This mixing can be performed by mechanical stirring, for example using a mechanical stirrer provided with a three-blade propeller. The rotation speed is, for example, 200 rpm, and the stirring time is, for example, 3 to 5 minutes.

The addition of the ferromagnetic compound to the solvent or to the mixture of the solvent and the component(s) mentioned above can be carried out by simply pouring the ferromagnetic compound into said mixture. When the ferromagnetic compound is added, the mixture containing the solvent, this ferromagnetic compound and, optionally, the component(s) mentioned above is generally kept under mechanical stirring.

This stirring can be carried out, for example, by means of a mechanical stirrer provided with a three-blade propeller. Stirring is continued until a homogeneous suspension is obtained.

The inorganic viscosifier(s) is then added, preferably gradually, to this homogeneous suspension, still under stirring.

The stirring speed is generally increased gradually as the viscosity of the solution increases, finally reaching a stirring speed of between 400 and 600 rpm, for example, when all the inorganic viscosifier(s) have been added, without any splashing.

Once the inorganic viscosifier(s) (mineral(s)) have been added, stirring is still continued, for example for 2 to 5 minutes, to obtain a perfectly homogeneous gel.

The gel prepared in this way is then left to rest for at least one hour before use.

It is clear that other protocols for preparing the gels used in the method according to the invention can be implemented with the addition of the gel components in an order different from that mentioned above.

Generally, the gel implemented in the method according to the invention should have a viscosity of less than 200 mPa·s under a shear of 1000 s⁻¹ so that it can be deposited or sprayed, nebulised or atomised in the form of fine droplets as defined above.

The viscosity recovery time should generally be less than or equal to one second and the low shear viscosity greater than 10 Pa·s so that it does not flow on a partition when deposited or sprayed with a thickness of less than 1 mm.

The ferromagnetic gel prepared in this way can be used in a method for decontaminating a surface or in a method for decontaminating a volume of a gaseous medium.

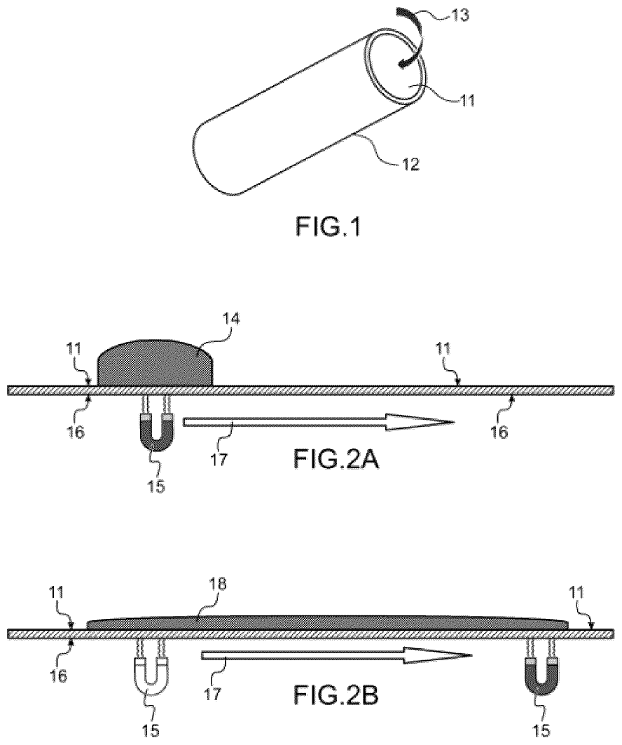

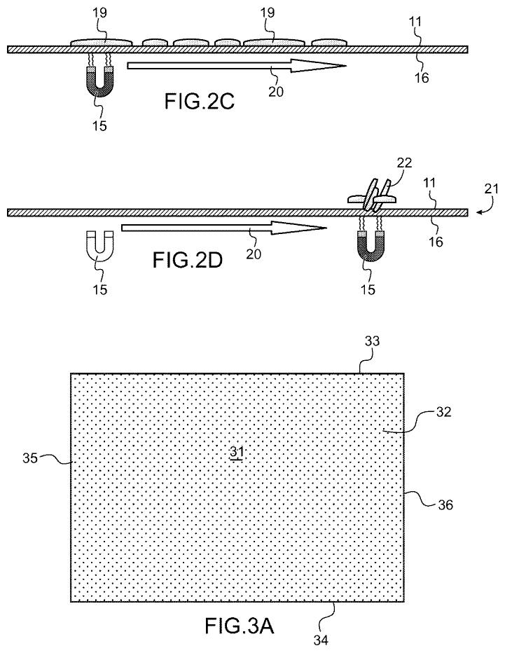

FIGS. 1, 2A, 2B, 2C and 2D illustrate a method for decontaminating the inner surface (11) of a duct, pipe (12) using a ferromagnetic gel containing ferromagnetic particles. This surface is not accessible to a gel applied by spraying or trowelling.

To begin with (FIGS. 1 and 2A), a sufficient amount of ferromagnetic gel (14) is deposited at the inlet (13) of the duct on the inner surface (11) of the duct (12) to be treated.

A magnet (15) is then placed close to the outer surface (16) of the duct (12).

The magnet is then moved (arrow 17 shows the movement of the magnet) (FIG. 2B) along the outer surface (16), causing the gel (14) to spread at a distance on the inner surface (11) of the duct (12). This forms a millimetric layer (18) of gel on the inner surface (11) of the duct (12). This layer (18) of gel is left on the inner surface (11) of the duct (12) for the time required to decontaminate the inner surface (11).

Drying the gel layer (18) is then performed, whereby dry gel solid residues (19) are formed on the inner surface (11) of the duct (12) (FIG. 2C).

Once the gel has dried and the inner surface (11) of the duct (12) has been decontaminated, the magnet (15) can again be used to recover the solid dry gel residues (19) from the inner surface (11) of the duct (12).

For this, the magnet is moved (arrow 20 shows the movement of the magnet) (FIGS. 2C and 2D) along the outer surface (16) of the pipe. The dry gel solid residues (19), which still contain the ferromagnetic particles, are then attracted by the magnet (15) and moved towards an outlet (21) of the duct (12) where they are gathered (22) and can be recovered (FIG. 2D).

At the end of the step illustrated in FIG. 2D, the inner surface is clean and decontaminated.

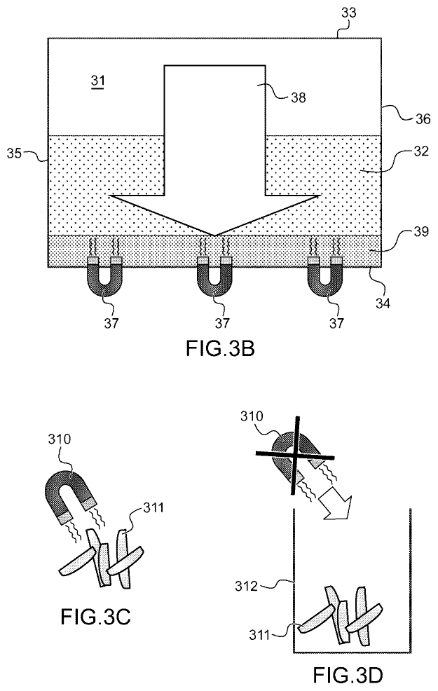

FIGS. 3A and 3B illustrate a method for decontaminating a volume of a gaseous medium or atmosphere using a ferromagnetic gel containing ferromagnetic particles.

The ferromagnetic gel is sprayed in the form of a mist of droplets to take up aerosols of contaminant species suspended in a volume of a gaseous medium such as air.

FIG. 3A shows the volume (31) containing the ferromagnetic gel droplets (32) sprayed therein, this volume is defined by a top partition (33), a bottom partition (34) and side partitions (35 and 36) (the other two side partitions are not shown in FIGS. 3A and 3B).

The droplets (32) of gel will take up the aerosols of contaminant species and pull them back onto the partitions (33, 34, 35, 36) of the volume (31).

One or several magnets (37) are used to speed up the decontamination operation or to move the gel droplets (32) within the volume (31) to be decontaminated. In FIG. 3B, three magnets (37) are implemented. The droplets of gel suspended which have taken up the contaminant species will then be attracted by the magnets and move into the zone where the magnets are located.

In this way, it is possible, on the one hand, to accelerate the fall of some very fine droplets which may remain suspended for several hours and, on the other hand, to define the zone in which the droplets will be able to agglomerate and dry to finally form a dry, solid residue containing the aerosols of contaminant species initially present in the volume.

In FIG. 3B, the magnets (37) are positioned in the vicinity of the bottom partition (34) and the suspended droplets are attracted by the magnets towards the bottom partition (34). The arrow (38) shows the movement of the suspended droplets attracted by the magnets. The droplets are thus pulled back downwardly more quickly, and will agglomerate and dry on the bottom partition (34) to form a dry, solid residue (39) on this partition.

FIGS. 3C and 3D show the recovery, using an electromagnet, of the dry, solid waste obtained after a decontamination operation carried out in accordance with the method according to the invention, using a ferromagnetic gel.

The use of an electromagnet allows easy recovery of the dry, solid waste containing the contamination and the ferromagnetic compound.

Firstly, the electromagnet (310) is activated to attract the waste, dry and solid residues (311) onto the surface and recover them (FIG. 3C).

The electromagnet can then be moved without any risk of spreading the contamination as it is contained within the magnetised solid residue.

Once placed over a suitable container (312), the electromagnet (310) can be deactivated (deactivation is shown by a black cross) and the solid residues (311) fall into the container (312) which can finally be dedicated to waste.

The invention will now be described with reference to the following examples, given for illustrative and non-limiting purposes.

### EXAMPLES

### Example 1: Preparation of Ferromagnetic Gels Implemented in the Method According to the Invention, and of Comparative Gels

This example describes the preparation of ferromagnetic gels implemented in the method according to the invention, and of comparative gels used in examples 2 to 6.

The ferromagnetic gels implemented in the method according to the invention (3, 4, 9, 10, 12 and 13) consist of a liquid, a gelling, viscosifier agent and ferromagnetic particles, while the comparative gels (1 and 2) consist solely of a liquid and ferromagnetic particles. Example 3 shows that the comparative gels 1 and 2, which do not comprise a viscosifier such as alumina or silica, do not fall within the scope of the ferromagnetic gels implemented in the method according to the invention.

These gels are prepared according to the following protocol:

The liquid, that is deionised water or 40% nitric acid, is first weighed in a suitable container. A ferromagnetic compound, that is ferrite, is gradually added to the liquid with stirring to obtain a homogeneous suspension.

The viscosifier, that is alumina or silica, is in turn added gradually, still with stirring. Once all the components have been incorporated, a gel is obtained and left to stirring for a few minutes to homogenise the mixture as much as possible.

The ferrite is “Iron (II, Ill) Oxide powder” ferrite, Fe₃O₄ marketed by SIGMA ALDRICH®.

The alumina is Aeroxide® Alu C alumina marketed by EVONIK INDUSTRIES AG®, which is a pyrogenated alumina with a specific surface area of 100 m²/g (BET).

The silica is Aerosil® 380 silica, marketed by EVONIK INDUSTRIES AG®, which is a pyrogenated alumina with a specific surface area of 100 m²/g (BET).

The formulations of the gels thus prepared are given in Table 1 below:

TABLE 1
Formulation of the gels used in examples 2 to 6.

| | | Mass | Mass | Mass | Total mass |
|---|---|---|---|---|---|
| Gel | Mass percent | percent of | percent of | percent of | percent of |
| number | of liquid | alumina | silica | ferrite | solid phase |
| 1 | 50 (water) | 0 | 0 | 50 | 50 |
| 2 | 66.7 (water) | 0 | 0 | 33.3 | 33.3 |
| 3 | 47.7 (water | 23.8 | 0 | 28.5 | 52.3 |
| 4 | 41.5 (water) | 37.7 | 0 | 20.8 | 58.5 |
| 9 | 63.3 (water) | 20.9 | 0 | 15.8 | 36.7 |
| 10 | 60.2 (water) | 24.7 | 0 | 15.1 | 39.8 |
| 12 | 83.2 (water) | 0 | 6.9 | 9.9 | 16.8 |
| 13 | 76.2 (40% | 0 | 9.5 | 14.3 | 23.8 |
| | nitric acid) | | | | |

### Example 2: Rheological Properties of Ferromagnetic Gels Implemented in the Method According to the Invention

In this example, it is demonstrated that the ferromagnetic gels implemented in the method according to the invention have the necessary rheological properties:
to be spread on a surface at a distance using a magnet,
to hold without flowing on vertical surfaces onto which they have been deposited with a millimetric thickness.

For this, the gels should be:

Rheofluidifying: their viscosity should decrease under shearing so that the gels can flow.

Yield stress fluids: At rest, gels should not flow in order to hold onto vertical surfaces. They should therefore have a yield stress of a few Pa. The yield stress is the stress below which a gel does not flow (it then behaves like a solid) and above which it does flow (it then behaves like a liquid).

It should be noted that these rheological properties also allow the gels to be applied by spraying.

Various rheological measurements have been carried out using a TA Instruments DHR1 rheometer in rough plane/plane geometry.

### Demonstration of the Rheofluidifying Nature of the Ferromagnetic Gels Implemented in the Method According to the Invention.

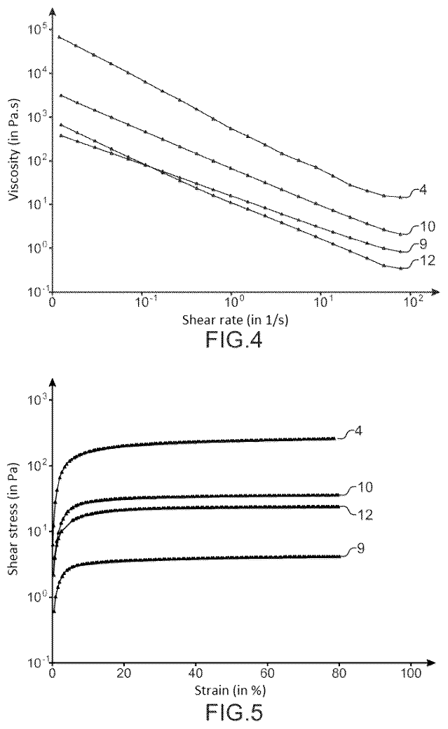

Firstly, the viscosity of the gels has been measured as a function of the shear rate. After pre-shearing for 1 minute at a shear rate of 20 s⁻¹, followed by 1 minute's rest, a continuous ramp of shear rates has been applied, ranging from 0.01 s⁻¹ to 100 s⁻¹, over a total period of 60 seconds. FIG. 4 shows the course of the viscosity (in Pa·s) of the ferromagnetic gels implemented in the method according to the invention as a function of the shear rate (in s⁻¹) for shear rates between 0.01 and 100 s⁻¹.

For each of the gels, a clear drop in viscosity with shear rate, which is characteristic of the behaviour of a rheofluidifying fluid, is observed in FIG. 4.

More particularly, it is observed that, for gels 4, 9 and 10, which are ferromagnetic gels implemented in the method according to the invention, comprising alumina as a viscosifier, the viscosity of the gels increases with the total mass percent of solid.

For gel 12 comprising silica as a viscosifier, a slightly smaller drop in viscosity is observed with the shear rate (the slope of the straight line is smaller).

All the ferromagnetic gels implemented in the method according to the invention therefore have a high viscosity at low shear (that is at rest) and a very low viscosity when sheared, which enables them to be spread under the effect of a magnetic field, or sprayed.

### Demonstration of the Yield Stress of the Gels in Example 1.

A low shear rate (0.00673 s⁻¹) is constantly applied to the gels in order to deform them from rest and thus determine their yield point. FIG. 5 represents the course of the shear stress (in Pa) of the gels as a function of the imposed strain (in %), at constant, low shear.

For each of the gels, it is noticed that, first of all, the stress sharply increases as a function of strain, so that the material is in the solid state (elastic strain). This is followed by a change in behaviour: the stress reaches the yield point and the material switches to liquid regime (stationary flow). The yield stress then corresponds to the stress at the gel yield point, the yield stress values obtained are given in Table 2.

TABLE 2
Yield stress values for gels 4, 9, 10 and 12 prepared in example 1.

| Yield stress | Yield stress | Yield stress | Yield stress |
|---|---|---|---|
| gel 4 (in Pa) | gel 9 (in Pa) | gel 10 (in Pa) | gel 12 (in Pa) |
| 190 | 3 | 30 | 21 |

Gel 4 has a high yield stress because its mass percent in the solid phase is fairly high. When the amount of solid phase in the gel formulation is reduced, the yield stress decreases (gel 10 then gel 9). As before, there is a change in behaviour when the viscosifier is modified. For example, gel 12 (with silica as the viscosifier) requires a lower mass percent of solid phase to achieve a significant yield stress of 21 Pa.

Within the context of surface decontamination operations, yield stress values greater than 10 enable the gels, once spread on surfaces using a magnet, not to flow and to adhere to them to a thickness of a few millimetres. Thus, of the gels in this example, gels 4, 10 and 12 can be used as magnetisable gels.

Within the context of atmospheric decontamination operations, such yield stress values enable the gels, once deposited onto surfaces after capturing contaminants, not to flow and to adhere to these surfaces over a thickness of a few millimetres.

In conclusion of this example, it appears that the magnetisable gels 4, 10 and 12 implemented in the method according to the invention do have the rheological properties required for remote application using a magnet for surface decontamination operations, but also for spraying in the form of fine droplets for atmospheric decontamination operations.

### Example 3: Spreading the Gels of Example 1 Using a Magnet

In this example, it is demonstrated that the spreading properties of the magnetisable gels of Example 1 depend on the presence in the gels of ferromagnetic particles and also of viscosifiers.

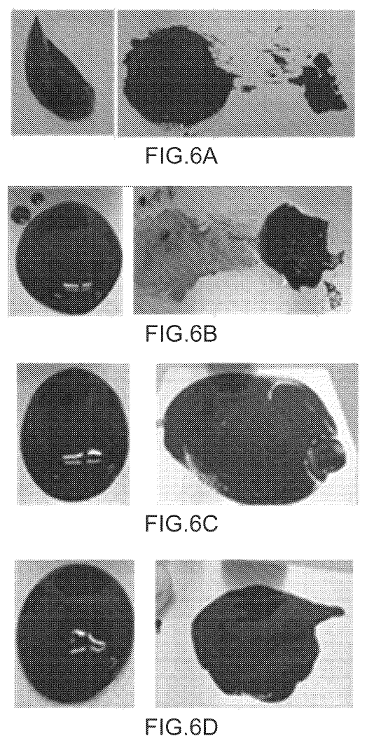

For this, gels 1, 2, 3 and 4 are first deposited onto an aluminium plate. A cylindrical magnet of type N829 from ECLIPSE MAGNETICS (neodymium magnet) is then used, which is moved manually over the opposite face of the aluminium plate in an attempt to spread the gel. FIGS. 6A, 6B, 6C and 6D, for gels 1, 2, 3 and 4 respectively, show the initial gel spot (left) as well as its shape after passage of the magnet (right).

For the comparative gels 1 and 2, which include no viscosifier (neither alumina nor silica), it is observed that the gels either move partially by fracturing (gel 1), or “slide” and do not adhere to the surface (gel 2). There is therefore no homogeneous spreading, which is explained by the absence of viscosifier to bind the formulation. This example clearly shows that the comparative gels 1 and 2 cannot be used within the framework of the method of the invention. Indeed, even if these comparative gels can be moved by magnetisation, they do not enable a millimetre thick gel layer to be deposited. For this, the presence of a viscosifier, such as alumina or silica, is necessary.

For ferromagnetic gels 3 and 4 implemented in the method according to the invention, which include a viscosifier (alumina): the gel spreads well as the magnet is moved and a certain millimetre thickness of gel adheres to the support as the rest is moved over the surface. The presence of the viscosifier helps to bind the formulation and therefore ensure satisfactory spreading. Furthermore, it is observed that gel 4, which has a higher mass percent of solid phase than gel 3, spreads less well. Indeed, when the same magnet is used, a more viscous gel is more difficult to spread. There is therefore a relation between rheological properties of the gel, strength of the magnet and ease of spreading.

### Example 4: Fracturing of the Gel on Drying and Recovery of Dry, Solid Waste

The ferromagnetic magnetisable gels implemented in the method according to the invention are gels with the properties of “suckable” gels, that is when they dry, they fracture and form dry, solid waste that can be easily recovered by brushing or sucking.

It is demonstrated in this example that the dry, solid waste obtained by drying the ferromagnetic gels implemented in the method according to the invention can also be recovered remotely, by magnetisation.

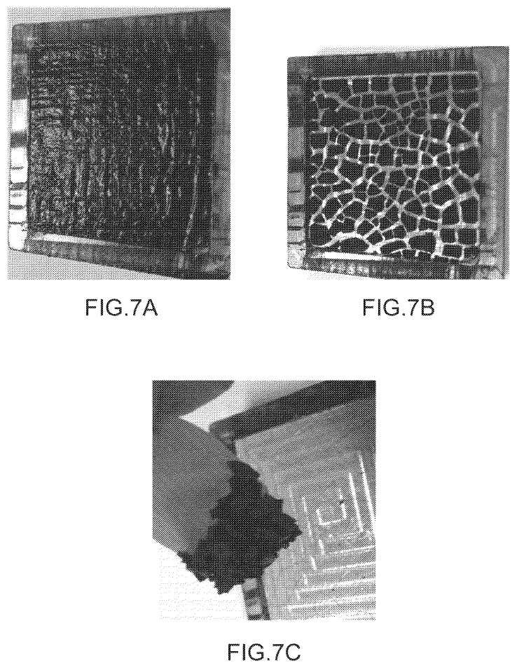

Tests in this example have been carried out in a 316L stainless steel pan. FIGS. 7A, 7B and 7C show the formation of solid waste after drying (at 25° C. and 40% relative humidity) (FIG. 7B) of a 1 mm thick layer of Gel 13 (76.2% nitric acid solution and 23.8% solid phase, see example 1) as well as the recovery of this waste using a magnet (FIG. 7C).

It is observed in FIG. 7A and FIG. 7B that drying a layer of Gel 13 results in the formation of a millimetre-sized solid waste. 2 g of gel have been spread out (FIG. 7A, before drying). After complete drying of the gel layer (FIG. 7B), the loss of mass is estimated at about 1.5 g. This means that approximately 75% of the gel mass had evaporated, that is almost all of the liquid phase.

In FIG. 7C, a magnet (protected by blue plastic) is used to recover the solid waste after drying. It is visually observed that all the waste has been recovered by magnetisation. After passage of the magnet, the residual amount of waste remaining adhered to the magnet is estimated at about 0.08 g (that is 4%). This waste comes from very thin layers on the edges of the pan. A sufficient thickness of gel is therefore required so that the waste generated can easily detach from the support and be attracted by magnetisation.

### Example 5: Corrosion Action of a Ferromagnetic Magnetisable Gel Implemented in the Method According to the Invention on Steel

In this example, the corrosion action of a ferromagnetic gel implemented in the method according to the invention on a surface is demonstrated.

Gel 13 (see example 1) is used to corrode a carbon steel surface.

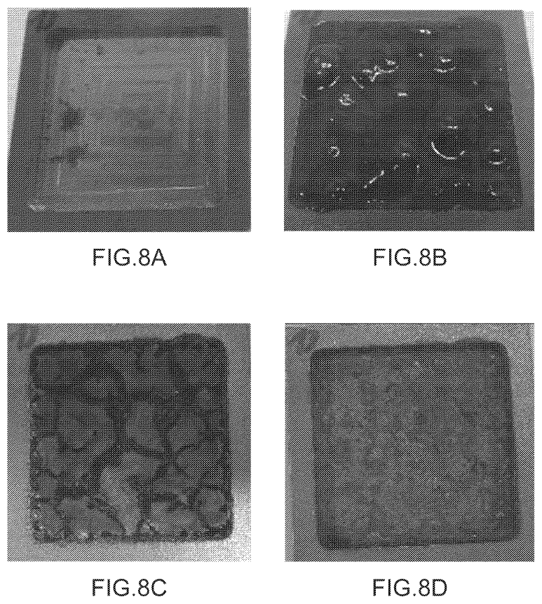

A thickness of 1 mm of Gel 13 (including 17.5% nitric acid) is deposited in a pre-weighed carbon steel pan. After drying, the gel flakes are removed and the pan is weighed again to estimate its mass loss. FIGS. 8A, 8B, 8C and 8D show the results obtained.

A clear degradation of the surface is observed after deposition and drying of the gel (FIG. 8D). Then, a mass loss of 0.5588 g is measured, which clearly demonstrates that magnetisable gels can corrode surfaces in the same way as suckable gels, and that they can therefore be used to decontaminate surfaces.

The corrosion thickness is estimated as follows:

The total surface area covered by the gel (bottom and edges of the pan) is 23.85 cm².

The density of the steel used is 7.8 g/cm³.

The corroded thickness is calculated as the ratio of mass/(surface area×density).

This gives an average corroded thickness of approximately 30 μm.

### Example 6: Decontamination of a Substrate Using Ferromagnetic Magnetisable Gels Implemented in the Method According to the Invention

In this example, the radioactive decontamination properties of ferromagnetic magnetisable gels implemented in the method according to the invention are demonstrated.

A caesium nitrate solution is prepared, and its concentration is determined to be equal to 2.86 mg·L⁻¹ by atomic absorption spectroscopy. Next, 2 mL of this solution are deposited at the bottom of a stainless steel pan. The solution is then allowed to evaporate. This simulates a surface contaminated with 5.72 mg of ¹³³Cs (simulating ¹³⁷Cs radioactive contamination).

A 1 mm layer of Gel 13 (see example 1) is applied to the artificially contaminated surface and the gel is left to dry to observe the formation of solid residues.

After drying, the solid residues are recovered and the surface is rinsed with water. The rinsing water is analysed by atomic absorption spectroscopy to determine the amount of Cs that has remained adhered to the support and, therefore, the effectiveness of the magnetisable gel.

After analysis, 0.58 mg of Cs has been measured in the surface rinse water. A Decontamination Factor (DF) can then be calculated, defined as DF=initial contamination mass/final contamination mass. A DF of approximately 10 is obtained for this test, which clearly demonstrates effectiveness of radioactive decontamination of a surface by a ferromagnetic magnetisable gel implemented in the method according to the invention.

### REFERENCES

[1] FAURE S., FUENTES P., LALLOT Y. “Gel aspirable pour la decontamination de surfaces et utilisation”: WO-A2-2007/039598.
[2] GOSSARD A., FRANCES F., LEPEYTRE C., “Gel de décontamination adsorbant et photocatalytique et procédé de décontamination de surface utilisant ce gel”: WO-A1-2018/011525.
[3] LUDWIG A., GOETTMANN F., FRANCES F., LEGOFF C., TANCHOU V., “Gel alcalin oxydant de décontamination biologique et procédé de décontamination biologique de surface utilisant ce gel”: WO-A1-2014/154818.
[4] GOSSARD A., FRANCES F., “Gel aspirable et procédé pour éliminer une contamination contenue dans une couche organique en surface d'un substrat solide”: WO-A1-2018/024990.
[5] GOSSARD A., TURC H. A., VENDITTI P., GRANDJEAN A., “Procédé de décontamination d'un milieu gazeux contaminé par des espéces contaminantes en suspension”: FR-A1-3083712.
[6] WO-A1-2014/154817.
[7] WO-A1-2013/092633.

What is claimed is:

1. A method for decontaminating at least one surface of a substrate of a solid material, said surface being contaminated with at least one contaminant species located on said surface and/or below said surface (subsurface) in the first layers of the substrate, wherein at least one cycle comprising the following successive steps is performed:
a) an inorganic ferromagnetic gel consisting of a colloidal solution comprising an inorganic viscosifier, a ferromagnetic compound and a solvent is applied to said surface, and then the gel applied to the surface is moved and spread at a distance using a magnet;
b) the gel is maintained on the surface at least for a time sufficient for the gel to destroy and/or inactivate and/or degrade and/or absorb the contaminant species, and for the gel to dry and form a dry, solid residue containing said contaminant species on the surface;
c) the dry, solid residue containing said contaminant species is moved and gathered on the surface using a magnet, and the dry, solid residue containing said contaminant species is recovered.

2. The method according to claim 1, wherein the substrate of a solid material is of a material selected from metals and metal alloys such as stainless steel, painted steels, aluminium and lead; polymers such as plastic materials or rubbers such as polyvinyl chlorides (PVC), polypropylenes (PP), polyethylenes (PE), in particular high density polyethylenes (HDPE), poly(methyl methacrylates) (PMMA), poly(vinylidene fluorides) (PVDF), polycarbonates (PC); glasses; cements and cementitious materials; mortars and concretes; plasters; bricks; natural or artificial stone; ceramics.

3. The method according to claim 1, wherein the gel is applied to the surface to be decontaminated at a rate of from 100 g to 2000 g of gel per m² of area, preferably from 500 to 1500 g of gel per m² of area, still more preferably from 600 to 1000 g of gel per m² of area, which generally corresponds to a thickness of gel deposited onto the surface of between 0.5 mm and 2 mm.

4. The method according to claim 1, wherein, during step b), a thickness of gel of from 2 mm to 2 cm is maintained on the surface using a magnet.

5. The method according to claim 1, wherein in step a), the gel is applied to the surface by spraying, brushing or trowelling, and then the gel applied to the surface is moved and spread at a distance using a magnet.

6. The method according to claim 1, wherein the surface is an inner surface of a pipe or duct, the gel is deposited onto the inner surface at the inlet of the pipe or duct, and is then moved and spread on the surface by means of a magnet placed in the vicinity of the outer surface or on the outer surface of the pipe or duct.

7. A method for decontaminating a volume of a gaseous medium contaminated with suspended contaminant species, said volume of a gaseous medium being in contact with at least one surface of a solid substrate, said method comprising the following successive steps:
a) an inorganic ferromagnetic gel consisting of a colloidal solution comprising an inorganic viscosifier, a ferromagnetic compound and a solvent is sprayed into said volume of a gaseous medium of fine droplets, thereby forming a mist;
b) the suspended contaminant species are captured, taken up by said droplets of ferromagnetic gel;
c) the droplets of ferromagnetic gel containing the suspended contaminant species captured are moved under the action of a magnet until they are deposited and accumulate on a determined zone of said surface of the solid substrate;
d) the gel is maintained on the determined zone of the surface of the solid substrate at least for a time sufficient for the gel to dry and form a dry, solid residue containing the suspended contaminant species captured;
e) the dry, solid residue containing said suspended contaminant species captured is recovered.

8. The method according to claim 7, wherein the volume of a gaseous medium is an enclosed volume defined by partitions, such as a floor, a ceiling and walls, forming said surface, the mist of fine droplets fills the entire enclosed volume, and the fine droplets of ferromagnetic gel containing the suspended contaminant species captured are deposited onto at least one of the partitions, preferably on a lower partition such as a floor.

9. The method according to claim 7, wherein the fine droplets have a size, defined by their largest dimension, such as a diameter, of from 1 to 1000 μm, preferably from 5 to 200 μm.

10. The method according to claim 7, wherein said suspended contaminant species are in the form of solid particles, liquid particles, or in the form of molecular species.

11. The method according to claim 1, wherein the contaminant species is selected, or the contaminant species are selected, from ionic, chemical, biological, nuclear or radioactive contaminant species.

12. The method according to claim 1, wherein the contaminant species is, or the contaminant species are, radioactive and/or chemically toxic and/or toxic contaminant species due to its (their) shape and/or size.

13. The method according to claim 12, wherein the contaminant species, or contaminant species, toxic due to its (their) shape and/or size, is (are) selected from contaminant species in the form of solid particles such as microparticles, or nanoparticles, for example in the form of fibres such as microfibres or nanofibres, in the form of nanotubes, or in the form of crystals such as nanocrystals.

14. The method according to claim 1, wherein the contaminant species is (are) selected from metals and metalloids in metal, metalloid or ionic form, preferably from so-called “heavy metals”, and toxic metals and metalloids in metal, metalloid or ionic form; compounds of these metals and metalloids, such as organometallic compounds, metal salts, metal oxides, metal carbides, etc. ceramics; wood; cereals; flour; asbestos; and glasses, for example in the form of glass wool.

15. The method according to claim 1, wherein the dry, solid residue is recovered using a magnet and/or by brushing and/or suction, for example with a suction device provided with a magnet.

16. The method according to claim 1, wherein the colloidal solution further comprises one or more component(s) selected from the following components:
a surfactant;
an active decontamination agent;
a getter for trapping gaseous contaminant species, in particular toxic or explosive gaseous contaminant species, such as hydrogen;
a superabsorbent polymer;
a contaminant species extractant.

17. The method according to claim 1, wherein the colloidal solution comprises, preferably consists of:
1% to 40% by mass, preferably 5% to 30% by mass, more preferably 5% to 25% by mass, still more preferably 8% to 20% by mass, relative to the mass of the gel, of at least one inorganic viscosifier;
0.1% to 40% by mass, preferably 5% to 30% by mass, relative to the mass of the gel, of at least one ferromagnetic compound;
optionally, 0.05% to 2% by mass, relative to the mass of the gel, of at least one surfactant;
optionally, 0.05 to 10 mol/L of gel, preferably 0.1 to 5 mol/L of gel, even more preferably 1 to 2 mol/L of gel, of at least one active decontamination agent;
optionally, 0.1% to 5% by mass, relative to the mass of the gel, of at least one getter to take up, trap gaseous contaminant species, in particular toxic or explosive gaseous contaminant species, such as hydrogen;
optionally, 0.05% to 5% by mass, preferably 0.05% to 2% by mass, relative to the mass of the gel, of at least one superabsorbent polymer;
optionally, 0.1% to 5% by mass, relative to the mass of the gel, of at least one contaminant species extractant;
and the remainder solvent, the amount of solvent being at least 20% by mass, relative to the mass of the gel.

18. The method according to claim 1, wherein the inorganic viscosifier is selected from metal oxides such as aluminas, metalloid oxides such as silicas, metal hydroxides, metalloid hydroxides, metal oxyhydroxides, metalloid oxyhydroxides, aluminosilicates, clays such as smectite, and mixtures thereof.

19. The method according to claim 16, wherein the surfactant is selected from surfactants having one or more of wetting properties, emulsifying properties and detergent properties; and mixtures thereof.

20. The method according to claim 16, wherein the surfactant is selected from the group consisting of alcohol alkoxylates, alkyl aryl sulphonates, alkyl phenol ethoxylates, block copolymers based on ethylene oxide and/or propylene oxide, ethoxylated alcohols, ether phosphates, ethoxylated acids, glycerol esters, imidazolines, quaternary ammonium salts (quats), alkanolamides, amine oxides, and mixtures thereof.

21. The method according to claim 1, wherein the ferromagnetic compound is selected from ferromagnetic metals, such as iron, cobalt and nickel; ferromagnetic alloys such as Heusler alloys, and alloys forming permanent magnets such as rare earth permanent magnets such as neodymium or dysprosium or cobalt permanent magnets; and ferrites.

22. The method according to claim 16, wherein the active decontamination agent is selected from bases such as sodium hydroxide, potassium hydroxide, and mixtures thereof; acids such as nitric acid, phosphoric acid, hydrochloric acid, sulphuric acid, hydrogen oxalates such as sodium hydrogen oxalate, and mixtures thereof; oxidising agents such as peroxides, permanganates, persulphates, ozone, hypochlorites such as sodium hypochlorite, cerium IV salts, and mixtures thereof; quaternary ammonium salts such as hexadecylpyridinium (cetylpyridinium) salts, such as hexadecylpyridinium (cetylpyridinium) chloride; reducing agents; and mixtures thereof.

23. The method according to claim 1, wherein the solvent is selected from water; organic solvents such as terpenes and alcohols; and mixtures thereof.
